(19) United States
(12) Patent Application Publication
(10) Pub. No.: US 2018/0085719 A1
(43) Pub. Date: Mar. 29, 2018

(54) CONDITIONING AND TREATING A FLUID FLOW

(71) Applicants: Steve LARNER, Slough, Berkshire (GB); Sharon THENG, Slough, Berkshire (GB); Mark ASTON, Slough, Berkshire (GB); Ian MAYOR-SMITH, Slough, Berkshire (GB)

(72) Inventors: Steve LARNER, Slough, Berkshire (GB); Sharon THENG, Slough, Berkshire (GB); Mark ASTON, Slough, Berkshire (GB); Ian MAYOR-SMITH, Slough, Berkshire (GB)

(73) Assignee: HANOVIA LIMITED, Slough, Berkshire (GB)

(21) Appl. No.: 15/563,510

(22) PCT Filed: Apr. 1, 2016
(86) PCT No.: PCT/GB2016/050943

§ 371 Date: Sep. 29, 2017

(30) Foreign Application Priority Data
Apr. 2, 2015 (GB) 1505803.5

(51) Int. Cl. B01F 5/06

(52) U.S. Cl. CPC B01F 5/0618; C02F 1/32

(57) ABSTRACT

A device for conditioning a fluid flow in a duct around an irradiating element such as an UV element, comprising means for generating multiple vortices in a fluid flow wherein the generating means comprises a flow conditioning body with an orifice, whereby the orifice shape is defined by an inner edge of the body arranged such that the distance from the centre of the orifice to the inner edge of the body varies substantially sinusoidally around at least part of the inner edge of the body.

The present invention relates to devices and methods for conditioning a fluid flow, preferably as part of an apparatus for treating a fluid flow. In particular, the present invention relates to the sanitisation of fluids, preferably using ultraviolet (UV) radiation.

A known method of treating fluid involves placing an ultraviolet (UV) radiation source (‘UV lamp’) in a duct, fluid conduit or chamber. A fluid to be treated is introduced into the duct and flowed past the UV lamp before exiting the duct. As the fluid flows through the duct, any pathogens within the fluid are exposed to UV radiation emitted by the UV lamp and thereby rendered harmless.

UV radiation is defined as electromagnetic radiation with a wavelength between 10 nm and 400 nm. Absorbed UV radiation produces photochemical reactions in organisms which stop an organism replicating, for example, by forming an additional bond between two adjacent thymine bases and therefore breaking the hydrogen bond linking the opposite Adenine bases to the Thymine bases in double-stranded DNA of a microorganism.

According to an aspect of the invention, there is provided a device for conditioning a fluid flow in a duct, comprising means for generating multiple vortices in a fluid flow wherein the multiple vortices have differential vorticity such as to cause a fluid flow at an outer region of the duct to have a greater dwell time in the duct than a fluid flow at an inner region of the duct.

According to another aspect of the invention, there is provided a device for conditioning a fluid flow in a duct, comprising means for generating multiple vortices in a fluid flow, wherein a vortex generated in a fluid flow at an inner region of the duct will have a higher vorticity than a vortex generated in the fluid flow at an outer region of the duct such that there is differential vorticity in the fluid flow, whereby to promote mixing in the fluid flow downstream of the device.

The generating means may comprise a flow conditioning body with an orifice having a shape configured to generate differential vorticity in a fluid flow as it passes therethrough.

According to another aspect of the invention, there is provided a device for conditioning a fluid flow in a duct, comprising means for generating multiple vortices in a fluid flow, wherein the generating means comprises a flow conditioning body with an orifice having a shape configured to generate differential vorticity in a fluid flow as it passes therethrough.

An advantage of the present invention is that it can create uniform turbulent conditions in fluid flowing through a duct, which reduces the average spread of particle velocities within the duct and hence increases dwell time, improves mixing and hence maximises exposure to the UV radiation. In particular, the invention provides a device for entraining a particle (or pathogen) within as uniformly chaotically random fluid flow as possible. The time for which a fluid is constrained in a duct (“dwell time”) may be increased by 40-50% compared to previously used arrangements.

Another advantage of the present invention is that it provides an increased efficiency in creating the required turbulent conditions while incurring very little head loss.

Another advantage of the invention is that it does not require a swirl vane at an inlet to a duct to achieve a turbulent flow, which arrangement in any case provides a limited degree of mixing and is typically complex and therefore expensive to manufacture.

The shape of the orifice may be configured such that a fluid flow encounters the body at two or more different points along a periphery of the orifice, thereby to generate the differential vorticity in the fluid flow. At least part of the or a periphery or profile of the orifice shape may be substantially in the shape of a waveform defined by one or more periodic oscillations comprising a plurality of peaks and troughs.

The orifice shape may be defined by an inner edge of the body arranged such that the distance from the centre of the orifice to the inner edge of the body varies substantially sinusoidally around at least part of the inner edge of the body, preferably between a minimum and maximum distance.

The minimum distance from the centre of the orifice to the body may be between 25% and 95%, preferably between 50% and 85%, more preferably between 60% and 75%, and more preferably about 67%, of the distance from the centre of the orifice to the outer edge of the body. The maximum distance from the centre of the orifice to the body may be between 50% and 99%, preferably between 65% and 96%, more preferably between 80% and 93%, and more preferably about 89%, of the distance from the centre of the orifice to the outer edge of the body.

The waveform may be characterised in that a distance from a peak of the waveform to an outer perimeter of the body is between one and five times greater, preferably between two and four times greater, and preferably substantially three times greater, than a distance from a trough of the waveform to said outer perimeter of the body. The waveform may be further characterised in that a distance from the outer perimeter to a centre of the body is between one and five times greater, preferably between two and four times greater, and preferably substantially three times greater, than a distance from the peak of the waveform to said outer perimeter of the body.

The entire orifice shape may be defined by a periodically oscillating waveform, preferably the waveform may be substantially sinusoidal.

There may be between two and thirteen peaks in the waveform, more preferably there may be between three and eleven peaks in the waveform, more preferably still there may be between five and nine peaks in the waveform. Even more preferably there may be seven peaks and/or seven troughs in the waveform. There may be an odd number of peaks and/or troughs in the waveform. There may be an equal number of peaks and troughs in the waveform.

The regions (or sections) of the body that define the troughs and peaks may also be described as baffle sections. The baffle sections may be defined by their respective heights. For example, the baffle sections may be described as high (peak) and low (trough) baffle sections, respectively.

The radii at the point of inflection of peaks and/or troughs (i.e. the radii of the baffle sections) may be defined by scaling the device diameter to fit a commensurate number of cycles of peaks and troughs in all pipe diameters from about 10 cm to about 35 cm. The diameter of the device may therefore be at least about 10 cm.

The device may be generally circular, such that the body can fit inside a chamber/pipe/conduit/duct having a corresponding cross-sectional shape. The orifice may be generally centrally located in the device. The device may be substantially planar. The generating means may be arranged to generate vortices having at least two different vorticities in a fluid flow. There may be a substantially constant relationship between the vorticity of each of the vortices generated.

The generating means may be arranged to generate at least one pair of vortices; preferably wherein the at least one pair of vortices has mutual vorticity and/or is a matching pair; more preferably wherein the generating means is arranged to generate multiple pairs of vortices having different velocity patterns and/or having different strengths.

As referred to herein, peaks and troughs of a waveform (that may define the shape of the orifice) may be defined with reference to the outer edge of the body (or plate), such that a peak is the maximum distance from the outer edge of the body and a trough is the minimum distance to said outer edge of the body.

The device may further comprise means for supporting an ultraviolet (UV) lamp. The means for supporting may comprise a holder arranged to provide a receptacle for an end of the UV lamp, the holder being attached to the device, preferably via a plurality of elongate members. The receptacle may be generally centrally located in the device and the elongate members may extend radially outward from the holder thereby to attach it to the device.

The body may be generally circular and/or may be generally centrally located in the body. The body may be substantially planar, (e.g. it may be a substantially flat plate having minimal width only to maintain mechanical integrity).

The device may further comprise means for locating and/or securing the device in a duct. The means for locating/securing the device may be one or more outwardly protruding projections provided on the outer periphery of the body. The orifice may be laser cut.

The device may be a baffle plate, which may be fabricated using a 3D printer. A machine readable map, or machine readable instructions, may be configured to enable a 3D printer to fabricate the device as described above.

According to another aspect of the invention, there is provided an apparatus for treating a fluid flow comprising a device as described above.

The apparatus may further comprise means for disinfecting a fluid, said means being arranged in the apparatus at least partially downstream of the device. The apparatus may further comprise means for cleaning the means for disinfecting when located inside the duct.

The means for cleaning may be arranged to wipe along at least part of the means for disinfecting. A lead screw may be provided having mounted thereon a threaded connector arranged to travel along the lead screw when relative rotation occurs, wherein the means for cleaning is attached to the threaded connector, whereby rotation of the lead screw causes the means for cleaning to travel along the means for disinfecting. The means for cleaning may be a substantially annular ring arranged to encircle the means for disinfecting. The lead screw may extend substantially the length of the means for disinfecting. The lead screw may be rotatably attached to the device.

The apparatus may further comprise means for conveying a fluid having a fluid inlet and a fluid outlet, wherein the device is disposed in the conveying means between the fluid inlet and the fluid outlet, whereby fluid entering via the fluid inlet will pass through the device before it exits via the fluid outlet. The device may be secured within the apparatus at a position proximal to the fluid inlet.

The conveying means may be a substantially cylindrical duct or chamber. The conveying means may have a substantially constant diameter along its length. The fluid inlet may be provided by an open end of the cylindrical duct. The fluid outlet may not be coaxial with the fluid inlet. The fluid outlet may be substantially the same size and shape as the fluid inlet and/or the duct. The fluid outlet may be provided on a side of the duct such that at least part of the apparatus is generally L-shaped.

The apparatus may further comprise a generally elbow-shaped inlet conduit connecting to the fluid inlet, such that the apparatus is generally U-shaped. The elbow-shaped inlet conduit may have a bend of around 90 degrees. Elbow-shaped inlet conduits are currently mandatory for UV treatment systems in certain countries, such as Germany and the US, for example. With an elbow-shaped inlet, fluid flow is faster around the outer (longer) bend, whereby, when entering through the inlet of a duct, the effect of the faster flow is to reduce the ‘dwell’ time that the fluid spends in the duct as it flows through. The enhanced mixing effect of the present invention advantageously negates the effect of an “elbow inlet”.

The apparatus may further comprise means for supporting a means for disinfecting a fluid, wherein the means for supporting may be integral with the device. The means for supporting may comprise a holder arranged to provide a receptacle for an end of the means for disinfecting a fluid, the holder being attached to the device via a plurality of elongate members. The receptacle may be generally centrally located in the device and the elongate members may extend radially outward from the holder thereby to attach it to the device.

The apparatus and, in particular, the means for conveying a fluid may further comprise an end plate provided at a distal end of the duct that opposes the fluid inlet. The end plate may be arranged to support a distal end of the means for disinfecting a fluid. The end plate may be arranged to support a distal end of the lead screw of a means for cleaning. The end plate may be removable.

The means for disinfecting may be an ultraviolet (UV) radiation source configured to emit radiation having a germicidal wavelength, and the apparatus may be a UV treatment chamber for the treatment of water and/or other suitable fluids.

According to another aspect of the invention there is provided an ultraviolet treatment chamber for the treatment of water and other suitable fluids, comprising an apparatus as described above wherein the means for disinfecting is an ultraviolet (UV) radiation source configured to emit radiation having a germicidal wavelength.

According to another aspect of the invention there is provided a method for conditioning a fluid flow in a duct, comprising generating multiple vortices in a fluid flow, wherein the multiple vortices have differential vorticity such as to cause a fluid flow at an outer region of the duct to have a greater dwell time in the duct than a fluid flow at an inner region of the duct.

According to another aspect of the invention there is provided a method for conditioning a fluid flow in a duct, comprising generating multiple vortices in a fluid flow, wherein a vortex generated in a fluid flow at an inner region of the duct will have a higher vorticity than a vortex generated in the fluid flow at an outer region of the duct such that there is differential vorticity in the fluid flow, whereby to promote mixing in the fluid flow downstream of the device.

The method may further comprise positioning in the fluid flow a flow conditioning body having an orifice with a shape configured to generate differential vorticity in a fluid flow as it passes therethrough.

According to another aspect of the invention there is provided a method for conditioning a fluid flow in a duct, comprising generating multiple vortices in a fluid flow by positioning in the fluid flow a flow conditioning body having an orifice with a shape configured to generate differential vorticity in a fluid flow as it passes therethrough.

The method may further comprise generating at least two vortices having different vorticity in a fluid flow. The method may further comprise generating at least two vortices having a relationship between their relative vorticity that is substantially constant in any fluid. The method may further comprise generating multiple velocity vortices in a fluid flow. The method may further comprise generating at least one pair of vortices in the fluid flow. The at least one pair of vortices generated may have mutual vorticity and/or are a matching pair.

The method may further comprise generating multiple pairs of vortices. The multiple pairs of vortices generated may have different velocity patterns and/or having different strengths.

The invention extends to a kit of parts comprising a plurality of devices, preferably of various configurations and/or sizes.

The invention extends to a method of designing the devices, preferably wherein the radii at the point of inflection of peaks and/or troughs in the waveform may be defined by the scaling of the device diameter such that the waveform comprises a commensurate number of cycles of peaks and troughs.

The waveform may be regular, which is to say periodic, or irregular or aperiodic. A composite waveform comprising the superposition of multiple waveforms may also be used. A composite of varying waveforms may be used at different points across the body.

As used herein, the term “sinusoidal” includes a sinusoidal or generally or curvilinear waveform arranged in a substantially circular configuration.

In some embodiments the waveform comprises a smooth continuous curve or plurality of curves, preferably without steps or other discontinuities.

As used herein, the term ‘baffle’ includes the definitions to deflect, check, restrain or regulate flow or passage (of a fluid). A ‘baffle’ may be a device or other means by which to baffle a fluid flow.

As used herein, the term ‘Vorticity’ includes the definition the ‘rate of rotation of a fluid at any point within a fluid flow’.

When a fluid is passing through any series of conduits, head loss within the fluid is undesirable but inevitable. The head loss is significant when a series of baffles are placed in the path of the fluid within a duct, and therefore requires additional energy to force a volume of fluid through the treatment process. By placing the device adjacent the inlet, a more thorough mixing of the fluid is achieved with a greatly reduced head loss. The apparatus therefore is less demanding of energy to treat the same volume of fluid than a similar apparatus using baffles throughout the duct treating the same volume of fluid.

Conventional practice also includes providing for the fluid as long a lead into the duct as possible, in order to ensure the highest possible Reynolds number, and hence turbulence, once the fluid passes across any devices or vanes within the duct itself. This requirement for a long, linear pipe to feed into the inlet of the duct limits where the purification system can be placed within a plant or factory.

The outlet conduit for the duct in the current state of the art is of a wider diameter than the duct, so as to reduce the velocity of the post-treatment flow. This serves to increase the dwell time of the fluid still undergoing treatment within the duct, but causes the purification apparatus to occupy a significant amount of expensive factory or plant real estate, as well as severely limiting where the apparatus can be placed. With the present invention, the fluid outlet is, preferably, the same size as the fluid inlet. The apparatus therefore has a smaller physical footprint, and less space is required to install and run it in a fluid treatment system.

Having a removable end plate allows easy access to the inner workings of the apparatus. Maintenance of the apparatus is therefore quicker and easier, saving time other associated costs of repair. The UV lamp can be easily accessed if replacement or repair becomes necessary and any build-up of detritus on the duct and fluid outlet can be more easily removed and the inner surfaces cleaned.

The apparatus may have a constant bore diameter; a conduit with a decreasing bore diameter produces jetting, which is undesirable.

The present invention can be used to treat various different fluids and is not limited to treating water. Other applications include the food and the waste industry, for example. The present invention may also be used in a pasteurisation process. Preferably, the fluid is a liquid.

The invention also provides a device as referred to and substantially described herein with reference to the accompanying drawings.

The invention also provides an apparatus as referred to and substantially described herein with reference to the accompanying drawings.

The invention also provides a method as referred to and substantially herein described with reference to the accompanying drawings.

Any apparatus feature as described herein may also be provided as a method feature, and vice versa. As used herein, means plus function features may be expressed alternatively in terms of their corresponding structure.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

### FIGURES

An exemplary embodiment of the present invention will now be described, by way of example only, with reference to the accompanying drawings, in which:

FIG. 1 shows an apparatus for treatment of a fluid flow;

FIGS. 2a and 2b show a device for conditioning a fluid flow;

FIG. 3 illustrates fluid flow through two distinct regions of a device;

FIG. 4 illustrates fluid flow through adjacent regions of device;

FIG. 5 is a sectional view showing an internal view of an apparatus;

FIG. 6 illustrates how vortex pairing can occur in a fluid flow; and

FIG. 7 is a graph showing a comparison of two different devices in an apparatus.

### DETAILED DESCRIPTION

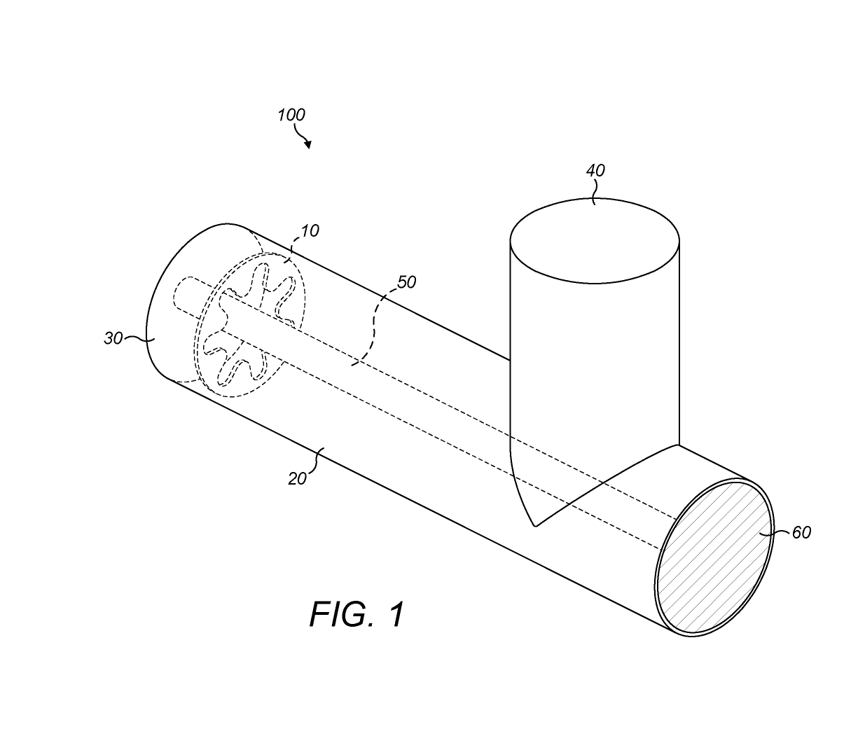

FIG. 1 shows an apparatus 100 for treating a fluid. The apparatus 100 comprises a duct (or “chamber”) 20 having a fluid inlet 30 and a fluid outlet 40 spaced apart such that at least part of the duct 20 provides a fluid conduit therebetween.

The duct 20 is generally cylindrical and has a substantially constant diameter (or ‘bore’) along its length. The inlet 30 and outlet 40 are also generally cylindrical, with both the inlet 30 and outlet having diameters roughly equal to the bore diameter of the duct 20. The outlet 30 is provided on a side of the duct 20 towards an end of the duct 20 that is distal to the inlet 30 such that the apparatus 100 is generally L-shaped. The end of the duct that is distal to the inlet 30 is closed.

Provided within the duct 20 is a means for treating (or sterilising) a fluid as it passes through the duct 20. In this embodiment, the means for treating a fluid is a UV lamp 50, which emits ultraviolet (UV) radiation. The UV lamp 50 is positioned within the duct 20 such that a fluid to be treated can flow around it, thereby being exposed to UV radiation for the duration that it flows past the UV lamp 50.

When using a UV lamp for disinfection, the UV radiation dose given to each pathogen (or ‘particle’) may vary according to the distance between the pathogen and the UV lamp and the exposure duration as the pathogen passes the UV lamp in the fluid flow.

The apparatus 100 is, ideally, arranged to be connected ‘in-line’ in a fluid treatment system such that a fluid to be treated enters the duct 20 via the fluid inlet 30 and exits the duct 20 via the fluid outlet 40.

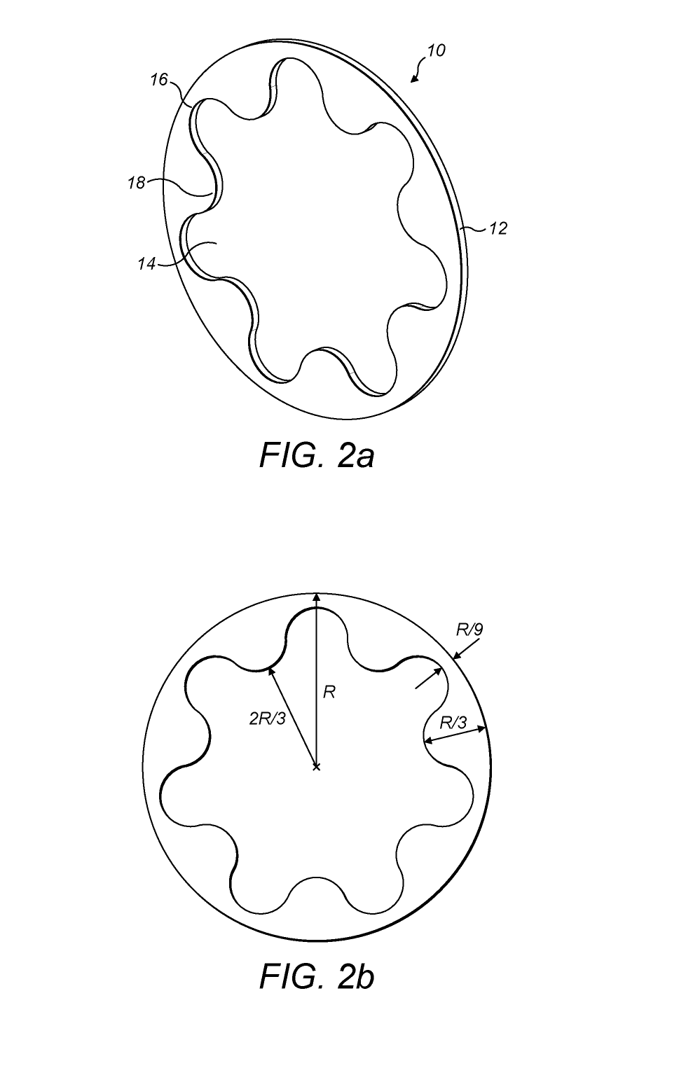

FIGS. 2a and 2b show a device 10 for conditioning a fluid flow, preferably for use with an apparatus 100 as shown in FIG. 1.

As shown in FIG. 2a, the device 10 comprises a substantially circular body 12 (or ‘plate’) that is, ideally, configured to fit within the duct 20 of the apparatus 100. The device 10 further comprises an orifice 14, ideally located generally centrally in the body 12.

The orifice 14 may generally be considered to have a shape defined by the internal radius (which extends from the centre of the body 12 to the edge of the orifice 14) of the device varying substantially sinusoidally around the perimeter of the orifice 14. The perimeter of the orifice may also be considered to represent a waveform (or a series of periodic oscillations) comprising alternating troughs 16 and peaks 18 having amplitudes that vary in a sinusoidal manner around the perimeter of the orifice 14. The distance of the troughs 16 and peaks 18 from the outer periphery (or edge) of the device body 12 may also vary sinusoidally between adjacent troughs 16 and peaks 18. The waveform, ideally, approximates a sinusoidal waveform, as discussed herein.

FIG. 2b shows a preferred embodiment, wherein the body 12 is configured to have an orifice with a shape defined such that the maximum distance from an outer edge of the body 12 to the edge of the orifice 14 (i.e. a peak 18) is roughly one third the distance to its centre (e.g. the radius of the body), and the minimum distance from the outer edge of the body 12 to the edge of the orifice 14 (i.e. a trough 16) is roughly one third of the maximum distance from the outer edge of the body 12 to the edge of the orifice 14 (i.e. the peak 18). A further way of expressing the minimum distance (i.e. a trough 16) in this preferred embodiment is that it is one ninth the distance from the outer edge of the body 12 to its centre.

Of course, a skilled person will recognise that the dimensions shown in FIG. 2b are only an exemplary embodiment of a configuration for a device 10. Said skilled person will understand that the relative radii between troughs 16 and peaks 18 and/or the number of troughs 16 and peaks 18 can vary while still maintaining the general arrangement that the distance from the centre of the body 12 to the aperture 14 (i.e. the radius to the perimeter of the orifice 14) varies substantially sinusoidally around the perimeter of the orifice 14 (i.e. inner edge of the body 12).

The device 10 has a substantially planar surface, preferably having a minimal thickness that provides it with a desired rigidity (and mechanical strength) to avoid it being deflected by a fluid flow. Ideally, the device may be considered to be two-dimensional.

The body 12 shown has a plurality of peaks 18 and troughs 16 that define the shape of the orifice 14. In the preferred embodiment shown, the orifice 14 has a shape defined by seven peaks 18 and seven troughs 16. The device 10 can easily be scaled up to fit a duct of larger diameter, preferably while retaining the same number of troughs 16 and peaks 18, of which the radii at the points of inflection will change accordingly.

The device 10 shown is generally circular, having a diameter configured to match an internal diameter of the duct 30 such that it fits inside the duct 30, as shown in FIG. 1.

An end plate 60 may be used to close off the distal end of the duct 20, past the outlet 40. The end plate 60 may be welded on to the end of the duct 20, or may preferably be removable, for example, be secured to the duct 20 via a bolted arrangement, to allow access to the interior of the duct 30.

The device 10 is located within the duct 20 in a substantially transverse orientation relative to the direction of fluid flow, preferably adjacent the fluid inlet 30 and upstream of the UV lamp 50. The device 10 ideally fits tightly within the bore of the duct 20 such that all of the fluid flowing through into the duct 20 flows through the orifice 14 of the device 10.

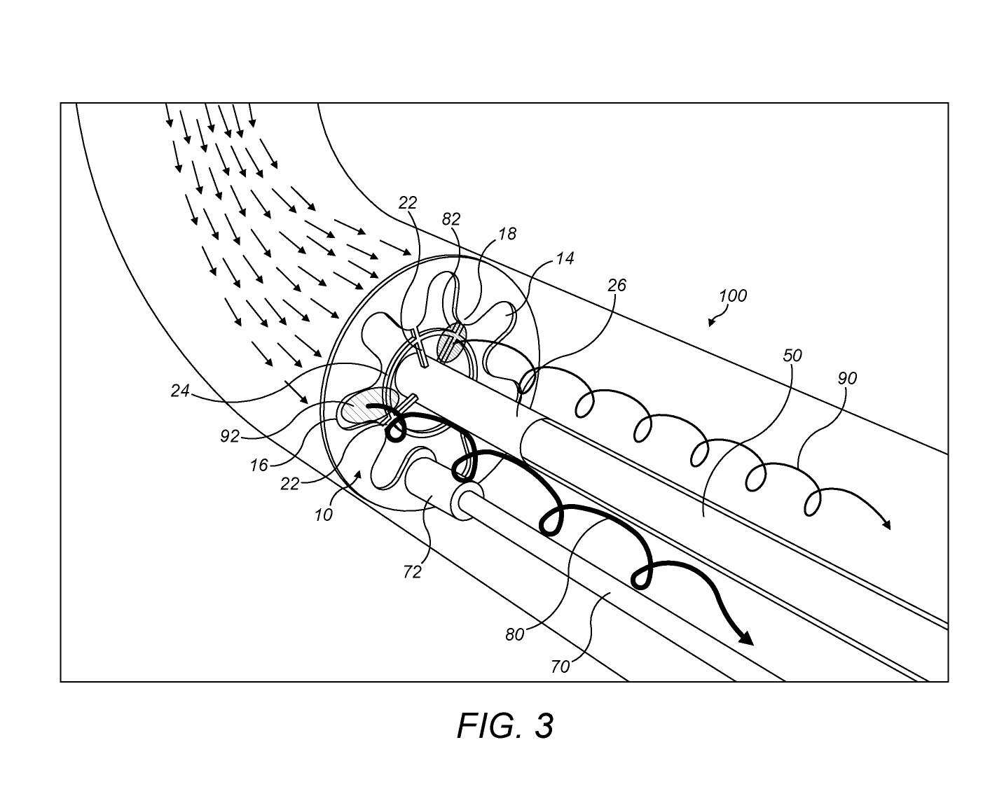

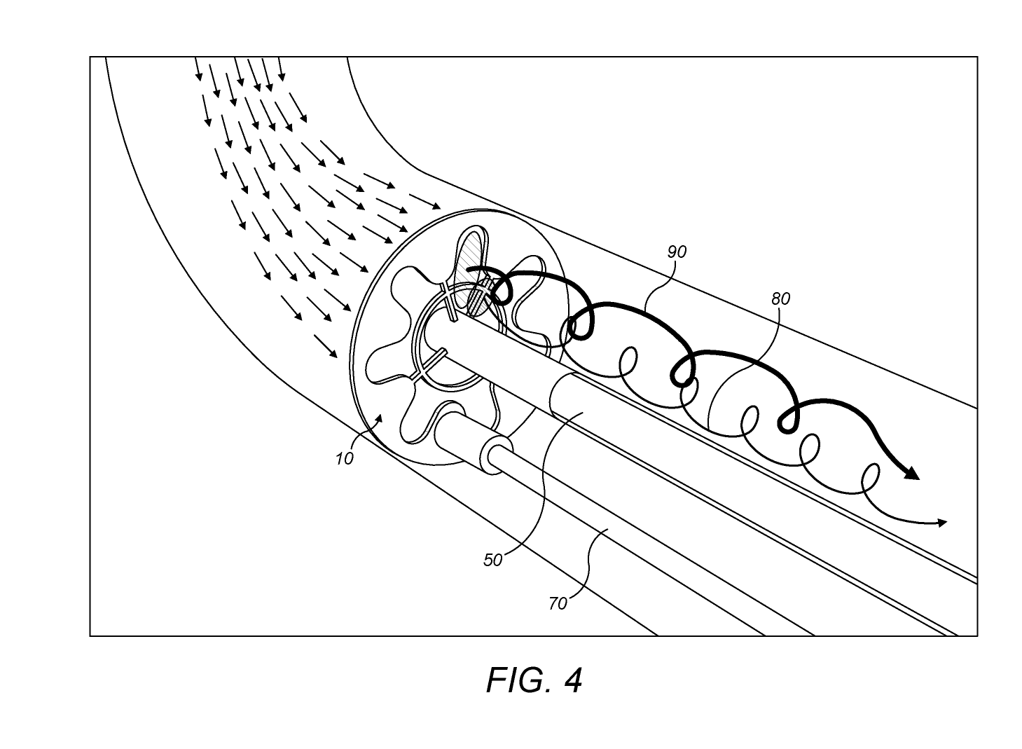

FIGS. 3 and 4 show schematic views of an apparatus 100 in use, with fluid flow lines 80, 90 illustrating two different types of fluid flow generated by the device 10.

The device 10 in FIGS. 3 and 4 is shown further comprising means 26 for supporting the UV source 50 within the duct 20 of the apparatus 100. The support 26 comprises a plurality of resiliently flexible elongate vanes (members) 22, which are configured in a peripheral arrangement about the holder 26 and extend radially outwards to the device body 12, where they connect with a corresponding peak 18 of the body 12 to support the holder 26 within the aperture 14. An end of the UV lamp 40 proximal to the inlet 30 is retained within and thereby supported by the ‘thimble-like’ holder 26, which fits tightly over the end of the UV lamp 50.

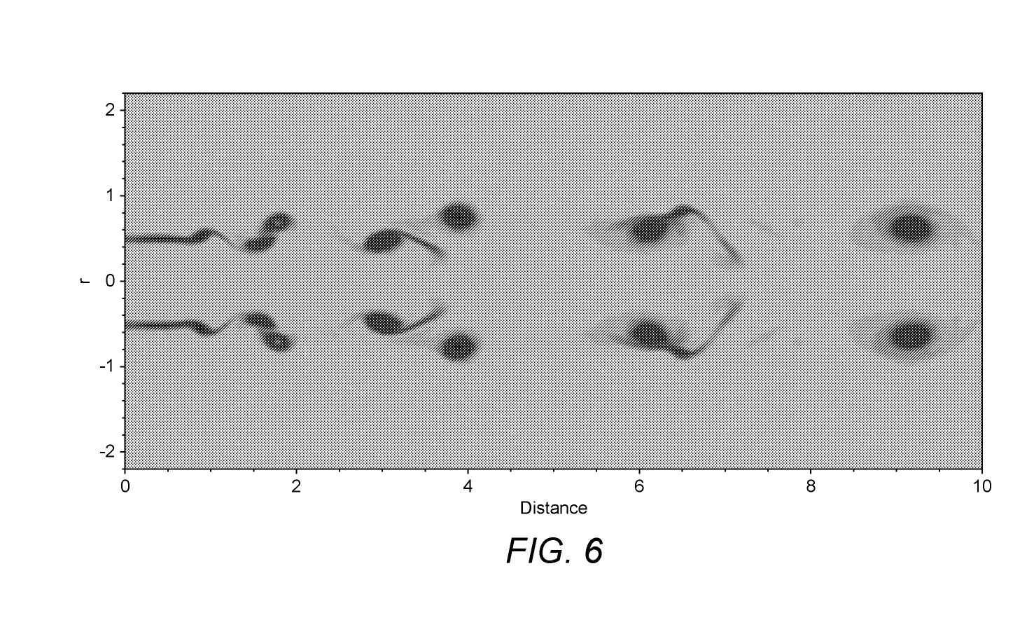

As fluid passes the through the orifice 14, either a trough 16 or a peak 18 generates eddies, vortices and other flow instabilities 80, 90 in the boundary layer of the fluid, and vortex pairing occurs (an example of this effect is illustrated in FIG. 6, discussed later). FIG. 3 illustrates the two vortices 80, 90 that are generated separately, each being generated by the fluid flow encountering the inner edge of the device body 12 at a trough 16 and peak 18 of the orifice 14 shape, respectively.

Fluid encountering the device body 12 at a peak 18 has a greater vorticity (and leaves the office at a higher velocity) than fluid encountering the device body 12 at a trough 16. This is due to an opening 82 for fluid to pass through between a peak 18 and the UV lamp holder 26 being smaller than an opening 92 provided for fluid to pass through between a trough 16 and the UV lamp holder 26. The smaller opening 82 between the peak 18 and the UV lamp holder 26 effectively causes ‘jetting’ in the fluid flow passing through the smaller opening 82, relative to the slower flow through the larger opening 92, which generates long trailing vortices. The larger and smaller openings 82, 92 alternate around the perimeter of the orifice 14, according to the waveform shape described above.

FIG. 4 shows two vortices generated by fluid flow passing through adjacent openings 82, 92 of the device 10. As the two vortices 80, 90 travel along the duct 30, they interact, with the higher vorticity vortex 90 effectively wrapping itself around the other vortex 80, with both vortices 80, 90 rotating in the fluid flow. The effect of this combined vorticity is that the fluid flow becomes turbulent with a Reynolds number (Re_{D})>4000), where D is the diameter of the duct 30.

In other words, the vortices 82, 92 provoke a mixing action within the fluid, increasing the Reynold's number and so the turbulence of the fluid. By generating multiple vortices 82, 92, the turbulence of the fluid is such that exposure to the UV lamp 50 is significantly more uniform, so that a statistical distribution of particle velocities in the fluid is narrowed such that a variance between the dosage amounts received by the pathogens (i.e. their exposure time to the UV radiation) within the fluid is minimised.

As the fluid flows through the orifice 14, vortices of different strength are generated in the fluid by the device 10. Fluid flowing through the duct 20 encounters different parts of the device body 12 along the orifice 14 periphery at different velocities. For example, fluid flowing at a relatively higher velocity in the flow will encounter the body at a peak 18 and generate relatively long trailing vortices whereas fluid flowing at a relatively lower velocity in the flow will encounter the body at a trough 16 and generate relatively large vortices. Thus, the device 10 produces multiple vortices of different velocity patterns, which interact causing chaos in multiple vortices thereby resulting in a greater dwell time in the fluid flow. A narrower range of distribution of particle velocities means all particles have similar exposure time to UV radiation emitting from the UV source 50.

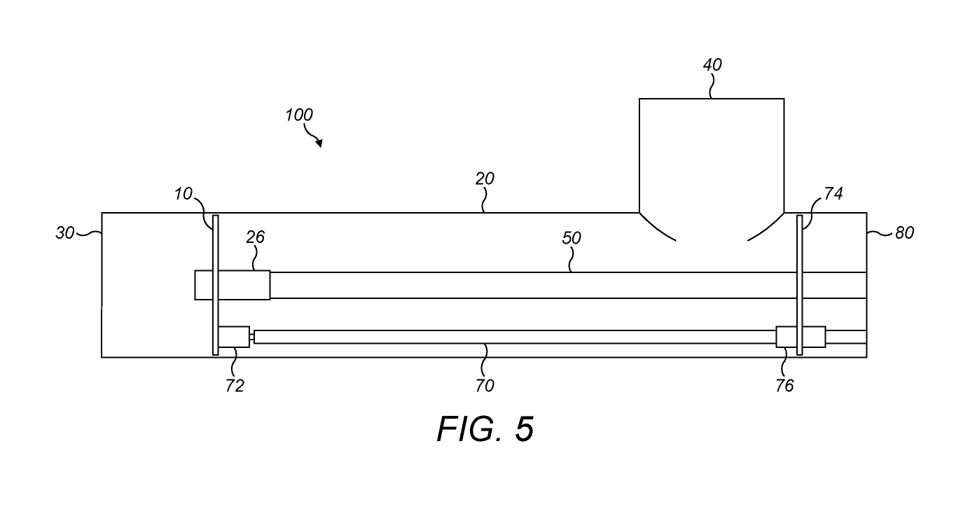

FIG. 5 shows a sectional side view of the apparatus 100. A means for cleaning the UV source 50 is provided within the duct 20, preferably comprising a cleaning attachment 74, preferably comprising neoprene, arranged to travel the length of the UV source 50 along a lead screw 70. Ideally, the cleaning attachment 74 is arranged to surround the UV lamp 50 and has negligible length relative to the UV source 50.

A first end of the lead screw 70 is secured to the device 10 via a socket 72, relative to which the lead screw 70 can freely rotate. An opposing second end of the lead screw may be received by the end plate 60 at the distal end of the duct 20. The lead screw 70 runs substantially parallel to the UV source 50, so that as the cleaning attachment travels along the lead screw 72 it wipes along the UV source 50.

The attachment 74 travels along the lead screw 70 by way of a connector 76 having an internal screw thread (not shown) corresponding to that of the lead screw 70. As the lead screw 70 is rotated (via an external input), the connector 76 (and hence also the cleaning attachment 74) travel along it by way of its internal screw thread.

As mentioned above, the attachment 74 makes physical contact with the UV lamp 50, preferably circumferentially enclosing it. The act of moving the attachment 74 therefore encourages the removal of sediment build up on the UV lamp 50. This provides an accessible and uncomplicated method for cleaning the UV lamp 50 when required. When cleaning is not required, the lead screw 70 can be rotated such that the attachment 74 is positioned at the distal end of the duct 20 from the inlet 10, so as not to disrupt the flow as far as possible.

The vorticity generated by the device 10 creates a sufficient dwell in the fluid around the UV lamp 50 to negate the accelerating effect that an ‘elbow’-shaped inlet conduit (not shown) has on the dwell time of a fluid flowing through the apparatus. An elbow-shaped inlet conduit, preferably having a roughly 90 degree bend, may be provided at the inlet 30 to an apparatus 100, to form a generally U-shaped apparatus 100 together with the outlet 40.

The differential between the different vorticity generated in fluid flows may be relatively constant, irrespective of the type of fluid. In other words, it is important that at least two different vortices are generated having different vorticity. It is the differential vorticity that causes the multiple vortices generated to interact due to their inherently non-stable, tumbling nature. The slower vortices have a wrapping effect about the faster, more chaotic vortices, which in combination may promote mixing in a fluid flow. Turbulence in the flow, and hence the differential vorticity is generated by the leading edges of the device body 12 at the edge of the troughs 16 and peaks 18 (“baffle sections”) at the periphery of the orifice (or aperture) 14.

The differential vorticity generated by the present invention in a fluid flow may be likened to a ‘tornadic’ effect in nature, whereby the pairing of two tornadoes of differential vorticity can occur. Vortex pairs may be generated in vortices and pair shedding may occur in the fluid flow downstream of the device 10. The more coherent flow will retain vortex pairs for longer than the chaotic flow, therefore shedding of vortex pairs will occur in the fluid flow at different points across the diameter of the duct of an apparatus, and hence at different distances from the device 10, with the biggest vortices shedding furthest from the device 10.

It is conceivable that an average UV radiation dose can be calculated by knowing the intensity of the UV lamp and the dwell time of the fluid within the duct, but without sufficient turbulence the distribution of UV radiation dosages received can be broad. Furthermore, it may be assumed that pathogens pass straight through the conduit past the UV lamp. Such an average measurement is therefore not a reliable indicator of whether a fluid has received sufficient disinfection treatment.

When the invention is used with an apparatus 100 as described herein, the multiple velocity vortices generated in a fluid flow by the device 10 may result in promotion of a spiral flow around the UV source 50, for example, when flow is through a UV treatment apparatus 100 and may further promote fluid flow about the UV source 50. Furthermore, the turbulence in the fluid can cause the flow of fluid away from the UV source 50 to stagnate. This provides the advantage of ensuring that, while the strength of the UV exposure received by particles in this slower flow occurring further from the UV source 50 may be less than that received by particles in the faster, more chaotic flow closer to the UV source 50, the duration of exposure to the stagnated particles is longer than for particles closer to the UV source 50. Thus, the statistical likelihood that all particles present in a fluid flow in a UV treatment chamber 100, for example, are exposed to a sufficient dosage of UV radiation to kill any pathogens is greatly increased. Furthermore, this allows the power output of the UV source 50 to be optimised.

FIG. 6 shows an example of vortex pairs being generated in a fluid flow. In this example, vortex pairing is shown occurring in a fluid flow as the distance of fluid flowing downstream of an orifice (having a radius “r”) increases. With specific reference to fluid dynamics, a vortex is a region in which flow rotates around an axis. Vortices make up a substantial component of turbulent flow. The vorticity of fluid in a vortex is higher in a region local to the axis of each vortex. As the distance from the axis is increased, the vorticity of the flow decreases in an inversely proportional manner to the distance from the axis.

Without multiple vortices being generated, the Reynold's number of the flow is smaller and so the flow may be more laminar, resulting in a broader range of particle velocities and hence exposure times to the UV lamp 50 and thus broader range of dosages of the disinfection treatment for any pathogens within the flow. A broad distribution of dosages received by pathogens within the flow is undesirable because some pathogens can receive a far higher dose than necessary, while leaving others relatively unscathed such that it is not possible to say with any certainly that the fluid has been thoroughly disinfected.

While a more powerful UV lamp 50 could be used to ensure that even the furthest pathogen flowing through a duct 20 receives a required dosage of UV radiation, this is undesirable as it is an unnecessary use of energy. Furthermore, the level of UV radiation emitted from UV lamps can be harmful to humans and therefore it is desirable to minimise the strength of the UV lamp 50. The generation of multiple vortices by the fluid passing through the orifice 14 within the device 10 narrows the distribution of dosages received by the pathogens within the flow. This makes it possible to predict with more accuracy the safety of a fluid following treatment and hence select an appropriately powerful UV lamp 50 (or other suitable disinfection source).

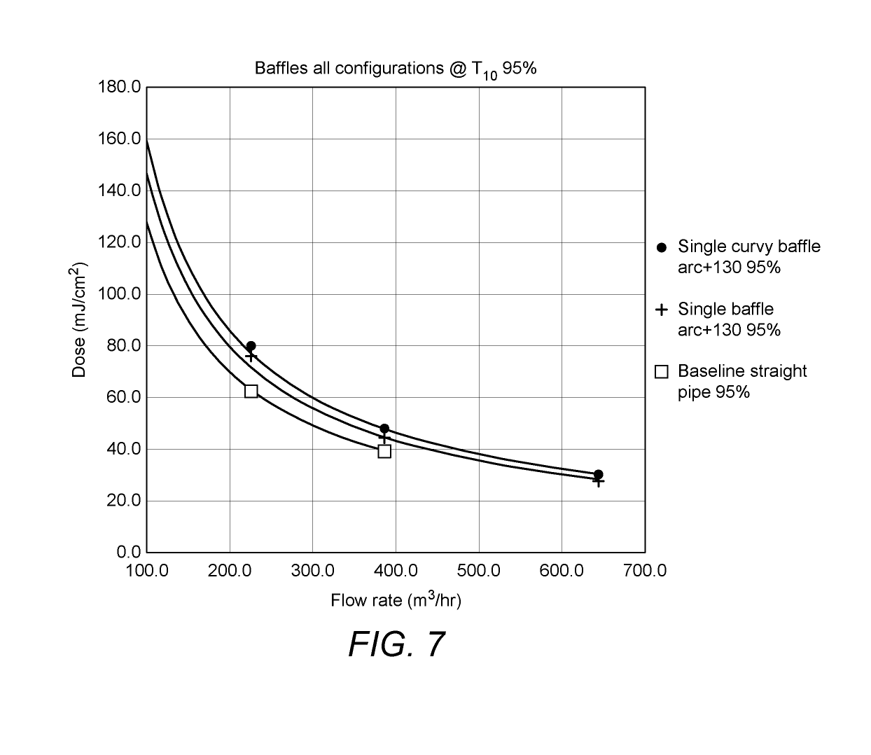

FIG. 7 is a graph showing the results of an exemplary test. The graph indicates the dose of UV radiation received per sq-cm (cm²) of an exemplary fluid at various flow rates, for three different device configurations. The fluid used to obtain these values was water having a T₁₀ value of 95%. The T₁₀ value, or ‘transmission value’, is a measure of the percentage of UV light that can pass through a 10mm distance within the fluid. A benchmark dosage for various flow rates is set using a straight duct 20 that does not contain a device for conditioning the flow of a fluid.

A single ‘curvy’ device 10 according to the present invention (e.g. having an aperture 14 shape defined by a substantially sinusoidal waveform, as described above), is then compared against a single ‘annular ring’ shaped device (not shown). It can clearly be seen that, tor each given flow rate for which data has been gathered, the fluid passing through the ‘curvy’ device 10 of the present invention received a higher dose of radiation (in mJ/cm²) than the fluid passing through either of the other two arrangements.

The device 10 is preferably fabricated from stainless steel, but can be formed from any material demonstrating the required physical properties to change the course of a fluid flow, including stainless steel, steel amalgams, high density polyethylene (HDPE), polytetrafluoroethylene (PTFE) or any another suitable plastic material. The duct 20, inlet 30, and outlet 40 can be formed from sheet metal pressed into the required shape, or any material demonstrating the required physical properties to contain a turbulent flow of the required flow rate. External ribs can be added to the outer wall to increase the rigidity of the duct 20.

Other methods of manufacture are possible and include sand casting, cold die compaction using stationary and rotary presses, cold and hot isostatic compression, powder injection moulding and selective laser sintering. The device 10, being a smaller component, is likely to be formed using metal injection moulding or cold isostatic pressing. The orifice 14 is formed by removing a section of material from the device 10 or from the material from which the device 10 is to be formed. This removal is preferably performed using laser cutting, although other methods are possible, for example using a jigsaw or an acidic compound.

In an alternative embodiment, multiple devices 10 may be placed throughout a duct 20 to generate additional vortices further downstream of an inlet. The one (or more) device 10 may be secured within a duct 20 using a suitable welding process, for example.

Other methods of manufacture may also be used. For example, the device may be manufactured by way of ‘3D printing’ whereby a three-dimensional model of the surface is supplied, in machine readable form, to a ‘3D printer’ adapted to manufacture the device. This may be by additive means such as extrusion deposition, Electron Beam Freeform Fabrication (EBF), granular materials binding, lamination, photopolymerization, or stereolithography or a combination thereof.

The machine readable model comprises a spatial map of the object or pattern to be printed, typically in the form of a Cartesian coordinate system defining the object's or pattern's surfaces. This spatial map may comprise a computer file which may be provided in any one of a number of file conventions. One example of a file convention is a STL (STereoLithography) file which may be in the form of ASCII (American Standard Code for Information Interchange) or binary and specifies areas by way of triangulated surfaces with defined normals and vertices. An alternative file format is AMF (Additive Manufacturing File) which provides the facility to specify the material and texture of each surface as well as allowing for curved triangulated surfaces.

The mapping of the surface may then be converted into instructions to be executed by 3D printer according to the printing method being used. This may comprise splitting the model into slices (for example, each slice corresponding to an x-y plane, with successive layers building the z dimension) and encoding each slice into a series of instructions.

The instructions sent to the 3D printer may comprise Numerical Control (NC) or Computer NC (CNC) instructions, preferably in the form of G-code (also called RS-274), which comprises a series of instructions regarding how the 3D printer should act.

The instructions vary depending on the type of 3D printer being used, but in the example of a moving printhead the instructions include: how the printhead should move, when/where to deposit material, the type of material to be deposited, and the flow rate of the deposited material.

The device as described herein may be embodied in one such machine readable model, for example a machine readable map or instructions, for example to enable a physical representation of said device or apparatus to be produced by 3D printing. This may be in the form of a software code mapping of the device and/or instructions to be supplied to a 3D printer (for example numerical code).

The invention described herein has many benefits for end users and the environment. For example, the increased hydraulic efficiency of a fluid treatment chamber incorporating the device arrangement offers 50% increase in energy efficiency for UV treatment; this has the benefit of reduced energy usage compared to conventional UV treatment systems and hence reduces operating costs (i.e. total lifetime cost to run), as well as reduced maintenance requirements and cost (i.e. lower UV lamp demand for increased component lifetime). This enabling technology therefore has the additional benefit of reducing total direct and indirect energy consumption, thereby reducing the carbon footprint for UV treatment systems.

A fluid treatment apparatus incorporating the device, as described herein, provides an additional beneficial effect in reducing the spread of possible UV dose exposure that bacteria and virus species experience. Due to the highly efficient mixing of particles in the flow, and the additional vortex-promoted mixing of fluid in the treatment chamber, the possibilities for particles to experience vastly differing exposures (e.g. whereby some particles travel near the treatment lamp, whilst others travel near the chamber wall away from the lamp) is reduced. This allows for a narrower statistical distribution of dose around the mean dose exposure. The nett effect is to reduce the full width, half maximum of the dose distribution curves compared to traditional UV treatment chambers.

The device is, substantially, a simple (almost) 2D shape that may be held in the chamber with bolts or other fixings for corrosive waters or fluids, hence facilitating easy service maintenance, or equally suited to hygienic welding process for market sectors where hygiene is ultra-critical, such as Food and Beverage, and Pharmaceutical industries; such industries likely use pure water in production process and hence do not require service of removable baffles. Allowing bespoke solutions for dedicated market applications makes the device a critically important step in underpinning its enhanced treatment capability in a wide range of UV treatment sectors.

It will be understood that the present invention has been described above purely by way of example, and modifications of detail can be made within the scope of the invention.

Each feature disclosed in the description, and (where appropriate) the claims and drawings may be provided independently or in any appropriate combination.

Reference numerals appearing in the claims are by way of illustration only and shall have no limiting effect on the scope of the claims.

1. A device for conditioning a fluid flow in a duct, comprising means for generating multiple vortices in a fluid flow wherein the multiple vortices have differential vorticity such as to cause a fluid flow at an outer region of the duct to have a greater dwell time in the duct than a fluid flow at an inner region of the duct.

2.-73. (canceled)
